(19) United States
(12) Patent Application Publication
(10) Pub. No.: US 2004/0034099 A1
(43) Pub. Date: Feb. 19, 2004

(54) Pharmaceutical composition

(76) Inventor: Beverly J. Ramsey, Durham, NC (US)

Correspondence Address: DAVID J LEVY, CORPORATE INTELLECTUAL PROPERTY, GLAXOSMITHKLINE, FIVE MOORE DR., PO BOX 13398, RESEARCH TRIANGLE PARK, NC, 27709-3398, US

(21) Appl. No.: 10/464,901

(22) Filed: Jun. 19, 2003

Related U.S. Application Data
(60) Provisional application No. 60/392,186, filed on Jun. 27, 2002.

(51) Int. Cl. A61K 31/195; A61K 9/20

(52) U.S. Cl. 514/567; 424/465

(57) ABSTRACT

The present invention relates to novel pharmaceutical compositions, in particular to compositions containing compounds of formula (I) as the active ingredient.

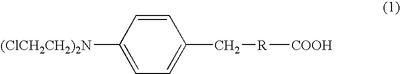

wherein R is an alkyleneamino or alkylene group.

The present invention relates to novel pharmaceutical compositions, in particular to compositions containing compounds of formula (I) as the active ingredient.

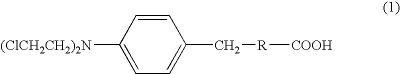

wherein R is an alkyleneamino or alkylene group. Two examples of compounds of formula (I) are melphalan and chlorambucil.

Melphalan is a well-established cytotoxic agent which is used to treat a range of neoplastic diseases including in particular multiple myeloma and ovarian cancer.

Melphalan has the chemical name 4-[bis(2-chloroethyl)amino]-L-phenylalanine and the structural formula:

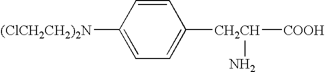

This compound is also known variously as L-phenylalanine mustard; L-PAM; L-sarcolysine. Melphalan is presently commercially available under the name Alkeran (TM, The Wellcome Foundation Limited) in the form of both injectable preparations and tablets.

Chlorambucil is a well-established cytotoxic agent which is used to treat a range of neoplastic diseases including chronic lymphocytic leukaemia, non-Hodgkins lymphoma and other refractory malignancies.

Chlorambucil has the chemical name 4-[bis(2-chloroethyl)amino]-benzenebutanoic acid and the structural formula:

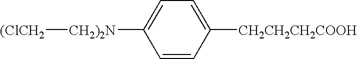

This compound is also known as 4-(4-bis(2-chloroethyl)aminophenyl]-butynic acid. Chlorambucil is presently commercially available under the name Leukeran (TM, The Wellcome Foundation Limited) in the form of tablets.

Typically prior art tablets contain melphalan and chlorambucil as the active ingredient and inactive ingredients such as lactose, magnesium stearate, erythosine, starch (e.g. potato starch, corn starch), povidone and sucrose. Optionally, these tablets are coated with a coating comprised of lactose, starch gelatine, and magnesium stearate in the case of melphalan or acacia, carnauba wax, polysorbate, starch, sucrose, talc, lactose, colouring, magnesium stearate and glaze in the case of chlorambucil (see tables 1a and 1b). Typically, these prior art tablets contained excipients of relatively high moisture content, in that, for example, corn starch generally contains 10-14% moisture, hydrous lactose can contain up to 5.5% moisture, pregelatinised starch can contain up to 14% moisture and is also hydroscopic and sodium starch glycolate can contain up to 10% moisture. Even though confectioner's sugar is a low moisture excipient in that it contains only about 1% moisture, it is hydroscopic.

In an attempt to improve the stability of such compositions, in particular tablets, a new composition has been formulated using low moisture excipients, which have preferably less than 7% moisture and most preferably 5% or less moisture.

The production of these prior art tablets involved wet granulation, blending and compression. The manufacturing process for the new products has been changed from wet granulation to a direct compression process, which is simpler, less time consuming, and easily contained. In addition to ease of manufacture, release of drug from the new products is expected to be superior to currently marketed tablets as the dissolution profiles for the new tablets demonstrate less tablet to tablet variability than those for the existing formulations.

The present invention therefore provides a novel, stable composition which comprises a compound of formula (I) as the active ingredient and excipients with low moisture content, in particular microcrystalline cellulose as the filler (diluent). The moisture content for microcrystalline cellulose is 5% or less.

Microcrystalline cellulose may typically be present between 20 to 98% by weight.

In particular the present invention provides a stable composition comprising melphalan or chlorambucil as the active ingredient. Melphalan and chlorambucil may be present between 1 to 5% by weight, preferably 2% by weight. Drug is released in a reproducible fashion from the tablets.

In addition, the composition according to the present invention preferably comprises amorphous fumed silica (silicon dioxide) as the glidant, typically at 0.1 to 0.5% by weight, preferably 0.25% by weight.

In a first embodiment of the present invention the stabilised composition comprises melphalan and microcrystalline cellulose. Preferably, it also comprises colloidal silicon dioxide, crospovidone and magnesium stearate. Crospovidone is present for adequate tablet disintegration and magnesium stearate is present as a lubricant.

In this first embodiment melphalan is typically present between 1 to 5% by weight, preferably 1.5 to 2.5%. Microcrystalline cellulose is present between 50 to 98% by weight, preferably more than 80% and most preferably about 96 to 97%. Colloidal silicon dioxide is present at 0.1 to 0.5% by weight, preferably 0.25% by weight and crospovidone may be present at 0.5 to 2% by weight, preferably 1%. Magnesium stearate may be present between 0.25 and 1.0% by weight, preferably 0.5%.

In a second embodiment of the present invention the stabilised composition comprises chlorambucil and microcrystalline cellulose. Preferably, it also comprises colloidal silicon dioxide, anhydrous lactose, and stearic acid. Stearic acid is present as a lubricant and anhydrous lactose is present as an additional filler. In this embodiment microcrystalline cellulose functions as both a filler and disintegrant.

In this second embodiment, the amount of chlorambucil present in the composition may be between 1 to 5% by weight, preferably 1.5 to 2.5%. Microcrystalline cellulose may be present between 20 to 40% by weight, more preferably between 25 and 35% by weight and most preferably about 29 to 30%. Anhydrous lactose may be between 60 and 80% by weight, preferably 65 to 75% and most preferably by 67%. Colloidal silicon dioxide may be present between 0.1 to 0.5% by weight, preferably 0.25% by weight. Stearic acid is typically present between 0.5 and 2% by weight, preferably 1%.

The moisture content of anhydrous lactose is less than 1%.

Preparation of the low moisture composition may comprise the following steps:

(a) blending

(b) compression

(c) coating suspension (optional)

(d) film coating (optional)

It should be noted that steps (a) and (b) are for tablet formation only with steps (c) and (d) being optional.

Accordingly, the present invention also comprises a method of preparation of a stabilised composition, particularly tablets, which comprises the above steps.

In addition to tablets, the composition according to the present invention may also be administered in the form of capsules, caplets, gelcaps, pills, and any other oral dosage forms known in the pharmaceutical art.

During blending all ingredients except magnesium stearate/stearic acid are added to a low shear mixer, such as a tumble blender and typically mixed for 20 to 40 minutes, preferably 30 minutes. The blend may be checked at this point for uniformity and further blended as necessary. The magnesium stearate or stearic acid is then added to the mixer and the powder blend lubricated for approximately 2 to 5 minutes, preferably 2 minutes.

During compression the lubricated blend is compressed on a suitable rotary tablet press. In-process controls for uniformity of weight, average weight and hardness, friability, and disintegration time are applied during the compression run and adjustments to the tablet press are made if necessary.

The coating suspension is prepared by adding water to a suitable mixing vessel, such as a stainless steel tank. In the case of Leukeran, a mixture of water and alcohol is used instead of water. Preferably the alcohol is ethanol and the ratio of water to alcohol is preferably 50:50 v/v. Chlorambucil polymerises in the presence of heat and moisture. The increased volatility of the hydro-alcoholic cosolvent allows the film-coating to be applied to the tablets at lower processing temperatures, producing tablets with lower levels of impurities resulting from the degradation of the drug product. The stirrer is turned on and Opadry^{R} White (Opadry^{R} Brown for Leukeran) is slowly added to the mixing vessel. The coating suspension is stirred until the Opadry is suitably dispersed based on visual examination.

Film coating is achieved by attaching the mixing tank containing the coating suspension to an appropriate spray pump. The tablets are charged into a perforated coating pan and warmed (with jogging) until a suitable exhaust temperature is achieved to allow the initiation of spraying. The suspension is sprayed onto the rolling tablet bed. A suitable exhaust temperature is maintained to ensure that the suspension is applied without over-wetting the tablet bed or exposing it to excess heat until the target application weight of dry coating solids has been sprayed (approximately 3 mg for a 100 mg tablet, giving an overall final weight of 103 mg). Upon completion of suspension application, the tablets are dried in a warm air stream. The tablets are then packaged in sealed amber glass bottles with plastic closures. Details of the processing conditions for the manufacture of Alkeran Tablets, 2 mg and Leukeran Tablets, 2 mg can be found in Examples 1 and 2, respectively.

Experiments to screen fillers, disintegrants, glidants and lubricants were conducted for the composition according to the present invention. Tablets were evaluated for physical characteristics and content of active ingredient at accelerated conditions (40° C./75% RH). The assay values were analysed statistically. The experiments demonstrated that the only filler that melphalan was sufficiently compatible with was microcrystalline cellulose, while chlorambucil was compatible with a mixture of anhydrous lactose and microcrystalline cellulose. Of all lubricants examined, chlorambucil was most compatible with stearic acid.

Summary of Characteristics of Stabilised Melphalan Formulation

After 24 months storage a stabilised composition according to the present invention including melphalan as the drug substance shows no significant changes in appearance. More preferably, there is no significant change in appearance after 36 months.

Moreover, the stabilised composition according to the present invention shows no significant changes in the level of active ingredient when stored at 25° C./60% RH in sealed bottles for 24 months. Typically, the active ingredient will have decreased by less than 10%, preferably less than 8% and more preferably 5% or less.

More specifically, in a stabilised composition according to the present invention, melphalan will have decreased by less than 10%, preferably 8%, more preferably 5% or even as little as 4% over a 24 month period when stored either at 25° C./60% RH or when stored over a 12 month period at 30° C./60% RH.

As far as drug related impurities are concerned a stabilised composition according to the present invention does not show an increase in levels of the monohydroxy derivatives of the melphalan upon storage for 36 months at 5° C.

The increase in melphalan dimer is 3% or less and preferably 2% or less upon storage for 36 months at 5° C.

The levels of individual principle unspecified impurities do not increase by more than 3%, preferably 2%, more preferably 1% and most preferably 0.5% when stored in sealed amber glass bottles for 36 months at 5° C.

The levels of total impurities increases by less than 3%, preferably less than 2% and most preferably less than 1.5% when stored for 36 months at 5° C.

As far as dissolution data for tablets comprising a stabilised composition according to the present invention is concerned, at least 60%, preferably 70%, most preferably 75% of the melphalan has dissolved at 30 minutes. When the tablets are stored for 36 months at 5° C., 75% and preferably 80% of the tablet has dissolved after 30 minutes.

See Example 3 for additional information regarding the stability of the stabilised composition of melphalan tablets.

Leukeran Stabilised Formulation

After 36 months storage a stabilised composition according to the present invention shows no significant changes in appearance.

Moreover, the stabilised composition according to the present invention shows less than a 5% decrease in the level of chlorambucil when stored for 24-36 months at 5° C./ambient humidity in sealed bottles. Typically, the chlorambucil content will have decreased by less than 10%, preferably less than 8% and more preferably 5% or less.

A stabilised composition according to the present invention, shows a decrease in chlorambucil content of less than 10%, preferably less than 8% and most preferably 5% or less when stored at 5° C./ambient humidity for 24-36 months and preferably less than 10% for up 36 months.

As far as drug related impurities are concerned a stabilised composition according to the present invention does not show an increase in levels of the monohydroxy derivatives of chlorambucil upon storage for 24-36 months at 5° C./ambient humidity.

As far as dimers and trimers chlorambucil are concerned, the increase over a 24-36 month period at 5° C./ambient humidity is less than 3%, preferably less than 2%. The increase in chlorambucil dimer is less than 3% and preferably 2% or less upon storage for 24-36 months at 5° C./ambient humidity. The increase in chlorambucil trimer and quadramer is less than 3%, preferably less than 2% and most preferably less than 1% upon storage for 24-36 months at 5° C./ambient humidity.

When stored at 5° C./ambient relative humidity the principle unspecified impurity content values are 1% or less, preferably 0.5% or less. The principle unspecified impurity content is not more than 1%, preferably 0.3% when stored at 5° C./ambient humidity over a 24-36 month period.

The levels of total impurities increases by less than 3%, preferably less than 2% and most preferably less than 1.5% when stored for 24-36 months at 5° C./ambient humidity.

As far as dissolution data for tablets comprising a stabilised composition according to the present invention is concerned, at least 60%, preferably 70%, most preferably 75% of the chlorambucil has dissolved at 30 minutes. At least 60%, preferably 70% and most preferably 75% have dissolved at 30 minutes, for tablets that have been stored for 24-36 months at 5° C./ambient humidity.

See Example 4 for additional information regarding the stability of the stabilised composition of melphalan tablets.

The following examples illustrate various aspects of the invention but should not be used to limit its scope.

### EXAMPLE 1

Description of the Manufacturing Process for the Alkeran (Melphalan) Tablets, 2 mg

Table A1 contains the new formulation for ALKERAN Tablets, 2 mg.

TABLE A1
Components and Composition of Stabilised ALKERAN Tablets, 2 mg

| | Quantity/Dosage Unit | |
|---|---|---|
| Component | mg/tablet | Function |
| Melphalan¹ | 2.00 | Active |
| Microcrystalline Cellulose | 96.25 | Diluent/Disintegrant |
| Crospovidone | 1.00 | Disintegrant |
| Colloidal Silicon Dioxide | 0.25 | Glidant |
| Magnesium Stearate | 0.50 | Lubricant |
| Opadry White YS-1-18097² | 3.0 | Film Coating |
| Purified Water³ | 22 | Suspending Agent |

¹Quantity of melphalan is adjusted to 100% label claim based on potency factor supplied by Quality Assurance.
²Applied as a 12% w/w suspension. Reflects a theoretical weight gain of 3%.
³Removed during processing.

1. Dispensing and Sieving

The appropriate quantities of Microcrystalline Cellulose, Colloidal Silicon Dioxide, and Crospovidone, are weighed, passed through an appropriately sized screen (typically 850 μm), and added into a stainless steel blending container. The appropriate quantity of Magnesium Stearate is weighed. The required quantity of melphalan is weighed and passed through an appropriately sized screen (typically 850 μm) into the blending container. Prior to lubrication of the blend, the Magnesium Stearate is passed through an appropriately sized screen (typically 850 μm) and added to the blender container.

The appropriate quantity of Opadry is weighed and placed in a sealed container.

2. Blending

The blender containing the Melphalan, Microcrystalline Cellulose, Crospovidone, and Colloidal Silicon Dioxide is mixed for approximately 30 minutes (range=20-40 minutes). The screened Magnesium Stearate is added to the blender and the powders are mixed for approximately 2 minutes (range=2-5 minutes).

3. Compression

The lubricated powder blend is compressed using 6.5 mm standard concave round tooling using a suitable rotary tablet press. In-process testing for average weight, uniformity of weight, average hardness, friability, and disintegration time are performed and adjustments made to the tablet press if necessary.

4. Preparation of Coating Suspension

Purified Water is weighed into a suitable mixing vessel, such as a stainless steel tank. The stirrer is turned on and the Opadry is slowly added to the mixing vessel. The coating suspension is stirred for at least 45 minutes to suitably disperse the Opadry, based on visual examination.

5. Film Coating

The mixing tank containing the coating suspension is attached to an appropriate spray pump. The tablets are charged into a perforated coating pan and warmed until the exhaust air temperature reaches a point suitable to initiate the spraying, approximately 46° C. The suspension is sprayed onto the rolling tablet bed. A suitable exhaust air temperature is maintained at approximately 46° C. (range: 42-50° C.), to ensure that the tablets do not become overly wet or hot during the coating process. The coating suspension is applied until the theoretical application weight of dry coating solids is reached within the range of 2.5-3% w/w, equivalent to the theoretical range of 2.5-3.0 mg per tablet. The typical target is a 3% theoretical weight gain (3 mg/tablet). This range of film-coating application is provided to allow the flexibility of stopping the film-coating process if necessary to minimise erosion of tablet engraving or prevent logo fill-in (bridging defects) as long as the theoretical minimum limit of film-coating suspension is applied (2.5% w/w, equivalent to 2.5 mg/tablet). Upon the completion of suspension application, the tablets are dried in a warm air stream providing an exhaust air temperature of approximately 46° C. (range 42-50° C.) for approximately 15 minutes (range 10-20 minutes).

### EXAMPLE 2

Description of the Manufacturing Process for the Leukeran (Chlorambucil) Tablets, 2 mg

Table A2 contains the new formulation for LEUKERAN Tablets, 2 mg.

TABLE A2
Components and Composition of Stabilised
LEUKERAN Tablets, 2 mg

| | Quantity | |
|---|---|---|
| | (mg)/Dosage | |
| Component | Unit | Function |
| Chlorambucil | 2.10¹ | Active |
| Microcrystalline Cellulose | 29.00 | Diluent, |
| | | Disintegrant |
| Lactose, Anhydrous | 67.65 | Diluent |
| Colloidal Silicon Dioxide | 0.25 | Glidant |
| Stearic Acid | 1.00 | Lubricant |
| OPADRY ® Brown YS-1-16655-A | 3² | Film Coating |
| Alcohol 96% | 9.8^{2,3} | Vehicle for Film |
| | | Coating Suspension |
| Purified Water | 12.2^{2,3} | Vehicle for Film |
| | | Coating Suspension |

¹Includes a 5% overage.
²Based on a 12% w/w hydro-alcoholic suspension and theoretical tablet weight gain of 3%.
³Removed during processing.

1. Dispensing and Sieving

The appropriate quantities of Colloidal Silicon Dioxide, Microcrystalline Cellulose and Lactose, Anhydrous, are weighed and passed through an appropriately sized screen (typically 850 μm) directly into a stainless steel blending container. The appropriate quantity of Stearic Acid is weighed.

The required quantity of chlorambucil is weighed and passed through an appropriately sized screen (typically 850 μm) into the blender container. Prior to lubrication of the blend the Stearic Acid is passed through an appropriately sized screen (typically 850 μm) and added to the blender container.

The appropriate quantity of Opadry is weighed and placed in a sealed container.

2. Blending

The blender containing the Chlorambucil, Microcrystalline Cellulose, Lactose, Anhydrous, and Colloidal Silicon Dioxide is mixed for approximately 30 minutes (range=20-40 minutes). The screened Stearic Acid is added to the blender and the powders are mixed for approximately 2 minutes (range=2-5 minutes).

3. Compression

The lubricated blend is compressed using 6.5 mm standard concave round tooling on a suitable rotary tablet press. In-process testing for average weight, uniformity of weight, average hardness, friability, and disintegration time are performed and adjustments made to the tablet press if necessary.

4. Preparation of Coating Suspension:

Purified Water, USP is weighed into a suitable Mixing vessel, such as a stainless steel tank. Alcohol (96%), BP is weighed and added to the vessel. The stirrer is turned on and the Opadry Brown, YS-1-16655-A is slowly added to the mixing vessel. The coating suspension is stirred for at least 45 minutes to suitably disperse the Opadry, based on visual examination.

5. Film Coating

The mixing tank containing the coating suspension is attached to an appropriate spray pump. The tablets are charged into a perforated coating pan and warmed until the exhaust temperature reaches a point suitable to initiate the spraying, at least 30° C. The suspension is sprayed onto the rolling tablet bed. A suitable exhaust temperature is maintained at approximately 32° C. (range=28-36° C.), to ensure that the tablets do not become overly wet or hot during the coating process. The coating suspension is applied until the theoretical application weight of dry coating solids is reached within the range 2.5-3% w/w, equivalent to the theoretical range of 2.5-3.0 mg per tablet. The typical target is 3% w/w theoretical weight gain (3 mg/tablet). This range of film-coating application is provided to allow the flexibility of stopping the film-coating process if necessary to minimise erosion of tablet engraving or prevent logo fill-in (bridging defects) as long as the theoretical minimum limit of film-coating suspension is applied (2.5% w/w, equivalent to 2.5 mg/tablet). Upon the completion of suspension application, the tablets are dried in a warm air stream of approximately 35° C. (range 30-40° C.) for approximately 15 minutes (range 10-20 minutes).

### EXAMPLE 3

Stability of Alkeran (Melphalan) Tablets, 2 mg

1. Appearance

No significant change in appearance was observed for those tablets that were stored up to 24 months at 25° C./60% RH, 12 months at 30° C./60% RH, 6 months at 40° C./75% RH, exposed to UV light, (>200 W-hr/m²), or fluorescent light (>1.2 megalux-hr.)

2. Melphalan Content by HPLC

Melphalan stability content show on average a 3.5% decrease in active ingredient content over a 24-month period when stored either at 25° C./60% RH or a 12-month period when stored at 30° C./60% RH. A slightly higher average decrease of 5.1% was observed for the accelerated storage condition of 40° C./75% RH for 6-months. Samples stored both protected and exposed to UV light showed no significant loss in melphalan content. Samples stored protected under fluorescent light showed no significant loss in melphalan content, while samples stored exposed to fluorescent light showed up to an 8.4% decrease in melphalan content

3. Drug Related Impurities Content by HPLC

3.1. Monohydroxymelphalan

When stored in sealed amber glass bottles, levels of monohydroxymelphalan do not increase upon storage for 24-months at 25° C./60% RH, 12-months at 30° C./60% RH or 6 months at 40° C./75% RH.

3.2. Melphalan Dimer

When stored in sealed amber glass bottles, the level of melphalan dimer, the primary degradation product increases upon storage. The initial level of melphalan dimer was typically less than 1.0%, based on melphalan content for the stability batches presented. Upon storage for 24 months at 25° C./60% RH, melphalan dimer showed an increase on average of 1.1%. Upon storage for 12 months at 30° C./60% RH, melphalan dimer showed an increase on average of 1.3%. Accelerated storage at 40° C./75% RH showed an average increase in melphalan dimer content of 1.8% over initial values.

3.3. Principal Unspecified Impurity

When stored in sealed amber glass bottles, levels of individual principal unspecified impurities do not significantly increase when stored for 24 months at 2500/60% RH, 12 months at 30° C./60% RH, and 6-months in closed containers at 40° C./75% RH.

3.4. Total Impurities

Levels of total impurities increase by an average of 1.0% when stored for 24 months at 25° C./60% RH, 1.1% when stored for 12 months at 30° C./60% RH, and an average of 2.3% when stored in closed bottles for 6 months at 40° C./75% RH. The majority of the increase is due to the increase in melphalan dimer.

4. Dissolution

Dissolution data for tablets, stored for 24 months at 25° C./60% RH and for 12 months at 30° C./60% RH, met the proposed specification of Q=80% dissolved at 30 minutes.

### EXAMPLE 4

Stability of Leukeran (Chlorambucil) Tablets, 2 mg

1. Appearance

No significant change in appearance was observed for those tablets that were stored up to 36 months at 5° C./Ambient Humidity.

2. Chlorambucil Content by HPLC

Chlorambucil content data show on average up to a 5% decrease in active ingredient content over the 36-month period when stored at 5° C./Ambient Humidity.

3. Drug Related Impurities Content by HPLC

3.1. Monohydroxychlorambucil

Levels of monohydroxychlorambucil do not increase upon storage for 36 months at 5° C./Ambient Humidity.

3.2. Chlorambucil Dimer

The level of chlorambucil dimer remained constant upon storage for 36 months at 5° C./Ambient Humidity.

3.3. Chlorambucil Trimer

The level of chlorambucil trimer, a degradation product increased slightly upon storage. Upon storage for 36 months at 5° C./Ambient Humidity, chlorambucil trimer showed a 0.1% increase.

3.4. Chlorambucil Quadramer

The level of chlorambucil quadramer, a degradation product showed a slight increase upon storage. The initial level of melphalan quadramer was typically less than 0.3%, based on chlorambucil content for the stability batches presented. Upon storage for 36 months at 5° C./Ambient Humidity, chlorambucil quadramer showed up to a 0.1% increase over initial levels.

3.5. Principal Unspecified Impurity

When stored at 5° C./Ambient Humidity, the initial and 36-month principal unspecified impurity content values were not more than 0.3%, based on chlorambucil label.

4. Dissolution

Dissolution data for tablets, stored for 36 months at 5° C./Ambient Humidity met the proposed specification of Q=75% dissolved at 30 minutes.

TABLE 1a
Alkeran Tablets: Comparison between prior art tablets and new stabilised tablets

| Old U.S. Composition | | Old U.K. Composition | Stabilised Composition | | |
|---|---|---|---|---|---|
| Composition | Per Tablet | Composition | Per Tablet | Composition | Per Tablet |
| Melphalan, USP | 2.0 mg | Melphalan, BP | 2.10 mg | Melphalan, USP | 2.00 mg |
| Confectioner's sugar NF | 51.0 mg | Lactose, BP/PhEur | 48.00 mg | Microcrystalline Cellulose, NF | 96.04 mg |
| Lactose Monohydrate, | 70.0 mg | Starches, BP/PhEur | 6.00 mg | Crospovidone, NE | 1.00 mg |
| NF | | | | | |
| Magnesium Stearate, | 1.0 mg | Erythosine E127/FD&C Red | 0.08 mg | colloidal silicon dioxide | 0.25 mg |
| NF | | No. 3, Aluminium Lake | | | |
| Povidone, USP | 2.0 mg | Povidone, BP | 0.45 mg | Magnesium Stearate, NF | 0.50 mg |
| Starch, Potato, Dried | 14.0 mg | Magnesium Stearate, | 0.30 mg | | |
| | | BP/PhEur | | | |
| Total Core weIght | 140.0 mg | Total Core WeIght | 56.93 mg | Total Core weight | 100.00 mg |
| Coating | | Coating | | Coating | |
| | | Lactose, BP/PhEur | 151.88 mg | Opadry White | 3.00 mg |
| | | Starches, BP/PhEur | 15.19 mg | | |
| | | Gelatine, BP | 3.04 mg | | |
| Coating weight | | Coating Weight | | Coating weight | |
| Tablet Weight | 140.0 mg | Total Tablet weight | 227.80 mg | Total Tablet Weight | 103.00 mg |

TABLE 1b
Leukeran Tablets: Comparison between prior art tablets and new stabilised tablets

| Old U.S. Composition | | Old U.K. Composition | Stabilised Composition | | |
|---|---|---|---|---|---|
| Composition | Per Tablet | Composition | Per Tablet | Composition | Per Tablet |
| Chlorambucil, USP | 2.1 mg | Chlorambucil, BP | 2.10 mg | Chlorambucil, USP | 2.10 mg |
| Confectioner's sugar NF | 38.5 mg | Lactose, BP/PhEur | 27.30 mg | Microcrystalline | 29.00 mg |
| | | | | Cellulose, NF | |
| Lactose Monohydrate, NF | 38.5 mg | Sucrose, BP/PhEur | 27.30 mg | Lactose, Anhydrous, NF | 67.65 mg |
| Magnesium Stearate, NF | 1.04 mg | Pregelalinized Maize Starch, BP | 0.35 mg | coltoidal silicon dioxide | 0.25 mg |
| Pregeistinized Starch, | 0.5 mg | Magnesium Slearale, OP/PhEur | 0.75 mg | Stearic Acid, NF/BP | 1.00 mg |
| Corn NF | | | | | |
| Total Core Weight | 80.6 mg | Total Core Weight | 57.80 mg | Total Care Weight | 100.00 mg |
| Coating | | Coating | | Coating | |
| Acacia, Powdered, NF | | Lactoae, BP/PhEur | 145.50 mg | Opadry Brown | 3.00 mg |
| Carnauba Wax, No. 1, | | Starches, BP/PhEur | 15.00 mg | | |
| Yellow, NF | | | | | |
| Pharmaceutical Glaze, NF | | Pregelatinized Maize Starch, BP | 8.00 mg | | |
| Polysorbale 60, NF | | Quinoline Yellow WS/D&C, Yellow No. 10 | 0.10 mg | | |
| Starch, Wheat, NF | | Magnesium Slearale, BE/PhEur | 1.50 mg | | |
| Sucrose, Liquid | | | | | |
| Talc, USP | | | | | |
| | | Coating Weight | 170.10 | Coating Weight | 3.00 mg |
| | | Total Tablet Weight | 227.90 mg | Total Tablet Weight | 103.00 mg |

1. A pharmaceutical composition which comprises a compound of formula (I) 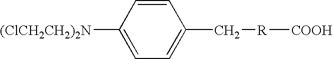
and microcrystalline cellulose.

2. A composition according to claim 1, wherein microcrystalline cellulose is present at between 20 to 95% by weight.

3. A composition according to claim 1, wherein melphalan is the active ingredient.

4. A composition according to claim 3, wherein melphalan will have decreased by less than 10% over a 36 month period when stored at 5° C./ambient humidity.

5. A composition according to claim 1, wherein chlorambucil is the active ingredient.

6. A composition according to claim 5, wherein chlerambucil will have decreased by less than 5% when stored for 36 months at 5° C./ambient humidity in sealed bottles.

7. A composition according to claim 5, wherein melphalan or chlorambucil is present at between 1 to 5% by weight.

8. A composition according to claim 5, wherein melphalan or chlorambucil is present at 2% by weight.

9. A composition according to claim 1 comprising colloidal silicon dioxide.

10. A composition according to claim 9, wherein the colloidal silicon dioxide is present at 0.1 to 0.5% by weight.

11. A composition according to claim 10, wherein the colloidal silicon dioxide is present at 0.25% by weight.

12. A composition comprising melphalan, microcrystalline cellulose, colloidal silicon dioxide, crospovidone and magnesium stearate.

13. A composition according to claim 12, wherein melphalan is present between 1 to 5% by weight, microcrystalline cellulose is present between 50 to 98% by weight, colloidal silicon dioxide is present at 0.1 to 0.5%, crospovidone is present at 0.5 to 2% by weight and magnesium stearate is present between 0.25 and 1.0% by weight.

14. A composition comprising chlorambucil, microcrystalline cellulose, colloidal silicon dioxide, anhydrous lactose and stearic acid.

15. A composition according to claim 14, wherein chlorambucil is present between 1 to 5% by weight, microcrystalline cellulose is present between 20 to 40% by weight, anhydrous lactose is present between 60 and 80% by weight, colloidal silicon dioxide is present between 0.1 to 0.5% by weight and stearic acid is present between 0.5 and 2% by weight.

16. A tablet comprising a composition according to claim 1.

17. A tablet according to claim 16 further comprising a film coating.
